(19) United States
(12) Patent Application Publication
(10) Pub. No.: US 2024/0399095 A1
(43) Pub. Date: Dec. 5, 2024

(54) SELECTIVELY ALTERING DEFORMATION CHARACTERISTICS OF A SYNTHETIC FABRIC MATERIAL

(71) Applicant: KONINKLIJKE PHILIPS N.V., EINDHOVEN (NL)

(72) Inventors: NATASHA A. GILBERT, PITTSBURGH, PA (US); LAUREN PATRICIA CHODKOWSKI, PITTSBURGH, PA (US); DANIEL STEED, NORTH HUNTINGDON, PA (US); JAMES ROBERT MAIER, PITTSBURGH, PA (US)

(21) Appl. No.: 18/804,163

(22) Filed: Aug. 14, 2024

Related U.S. Application Data
(63) Continuation of application No. 17/032,565, filed on Sep. 25, 2020.

(51) Int. Cl. A61M 16/06; B32B 37/16; D06C 23/00; D06C 29/00

(52) U.S. Cl. CPC A61M 16/0683; B32B 37/16; D06C 23/00; D06C 29/00; B32B 2305/20; B32B 2310/0843; D06C 2700/31; D10B 2331/02; D10B 2331/04

(57) ABSTRACT

A method of selectively altering one or more deformation characteristics of a polymer-based fabric material includes selectively melting a number of portions of the fabric in a predetermined pattern.

### CROSS-REFERENCE TO RELATED APPLICATIONS

This patent application is a Continuation of U.S. patent application Ser. No. 17/032,565, filed Sep. 25, 2020, which claims the priority benefit under 35 U.S.C. § 119 (e) of U.S. Provisional Application No. 62/906,757, filed on Sep. 27, 2019, the contents of which are herein incorporated by reference.

### BACKGROUND OF THE INVENTION

### 1. Field of the Invention

The present invention relates to methods of forming fabric materials and, more particularly, to methods of selectively stiffening fabric materials. The present invention further relates to fabric materials that have been selectively stiffened and to use of such materials in headgear for use in coupling a patient interface device to the head of a patient.

### 2. Description of the Related Art

There are numerous situations where it is necessary or desirable to deliver a flow of breathing gas non-invasively to the airway of a patient, i.e., without intubating the patient or surgically inserting a tracheal tube in the patient's esophagus. For example, it is known to ventilate a patient using a technique known as non-invasive ventilation. It is also known to deliver continuous positive airway pressure (CPAP) or variable airway pressure, which varies with the patient's respiratory cycle, to treat a medical disorder, such as sleep apnea syndrome, in particular, obstructive sleep apnea (OSA), or congestive heart failure.

Non-invasive ventilation and pressure support therapies involve the placement of a respiratory patient interface device including a mask component that is typically secured on the face of a patient by a headgear assembly. The mask component may be, without limitation, a nasal mask that covers the patient's nose, a nasal cushion having nasal prongs that are received within the patient's nares, a nasal/oral mask that covers the nose and mouth, or full face mask that covers the patient's face. It is known to maintain such devices on the face of a wearer by a headgear having one or more straps adapted to fit over/around the patient's head. Because such respiratory patient interface devices are typically worn for an extended period of time, it is important for the headgear to maintain the mask component in a desired position while doing so in a manner that is comfortable to the patient.

It is difficult to make versatile headgear that will fit all head shapes and sizes without compromising the seal of the mask. In-part, this is due to the difficulty in resolving conflicts between the need to be comfortable and the need to provide stability. An elastic headgear might be comfortable but also unstable. Conversely, a stiff headgear can stabilize a mask in place, but may compromise the comfort. This problem has been addressed to a certain extent by designing headgears to be high-stretch in some areas and low stretch in others through the use of different materials in each of such different areas. Such approach works well, but is limited by the fact that it involves making a headgear out of different materials that are cut and sewn together. The cut and sew method is not only generally expensive (due to the amount of materials and time needed) but also limits complexity of material properties on one piece of fabric. Accordingly, there is room for improvement in headgear for use in securing a mask to the head of a patient, as well as in the material or materials used in making such headgear.

### SUMMARY OF THE INVENTION

As one aspect of the invention, a method of selectively altering one or more deformation characteristics of a polymer-based fabric material is provided. The method comprises: selectively melting a number of portions of the fabric in a predetermined pattern.

The fabric material may comprise at least one of nylon or polyester fibers. Selectively melting the number of portions of the fabric in a predetermined pattern may comprise using a laser to melt the number of portions of the fabric material. Selectively melting the number of portions of the fabric in a predetermined manner may comprise chemically melting the number of portions of the fabric material. At least one portion of the number of portions may comprise a single fiber of the fabric material. At least one portion of the number of portions may comprise a plurality of fibers of the fabric material. The pattern may comprise a number of linear portions. The pattern may comprise a number of arcuate portions. The polymer-based fabric material may be one of a plurality of layers of a laminate material.

As another aspect of the present invention a polymer-based fabric material comprises one or more portions that have been melted in a predetermined pattern such that one or more deformation characteristics of the one or more portions have been selectively altered.

The fabric may comprise at least one of nylon or polyester fibers. The fabric material may further comprise a second layer of another material laminated thereto.

As yet another aspect of the present invention, a headgear for use in securing a patient interface device to the head of a patient comprises: a polymer-based fabric material wherein one or more portions thereof have been melted in a predetermined pattern such that one or more deformation characteristics of the one or more portions have been selectively altered.

The fabric material may comprise at least one of nylon or polyester fibers. The headgear may further comprise a second layer of another material laminated to the fabric material.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1 shows schematic representations of several different example fabric specimens having one dimensional stiffening patterns created in accordance with embodiments of the present invention;

FIG. 2 is a graph showing extension vs applied axial load for the several different example fabric specimens of FIG. 1;

FIG. 3A is a partially schematic view of a section of fabric material selectively stiffened in accordance with one example embodiment of the present invention;

FIG. 3B is a partially schematic view of another section of fabric material selectively stiffened in accordance with another example embodiment of the present invention;

FIG. 4A is a side view of a fabric headgear arrangement before being selectively stiffened in accordance with one example embodiment of the present invention, shown disposed on the head of a patient;

FIG. 4B is a side view of the fabric headgear of FIG. 4A after being selectively stiffened in accordance with one example embodiment of the present invention, shown disposed on the head of a patient;

FIG. 5A is a partially schematic view of a portion of fabric material selectively stiffened in accordance with one example embodiment of the present invention, shown in a relaxed positioning;

FIG. 5B is a partially schematic view of the portion of selectively stiffened fabric material of FIG. 5A, shown with an axial force applied thereto;

FIG. 6 is a partially schematic view of a portion of a fabric material selectively stiffened about an aperture defined therein in accordance with one example embodiment of the present invention;

FIG. 7A shows an unstiffened piece of fabric material having a circular aperture formed therein in a relaxed position and in a deformed position from an applied axial force;

FIG. 7B shows a piece of fabric similar to that of FIG. 7A and in similar conditions as FIG. 7A except with portions thereof selectively stiffened in accordance with one example embodiment of the present invention; and

FIG. 8 illustrates the deforming effect of varying stiffening patterns adjacent a circular aperture formed in pieces of fabric when an axial force is applied in accordance with exemplary embodiments of the present invention.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are coupled directly in contact with each other (i.e., touching). As used herein, “fixedly coupled” or “fixed” means that two components are coupled so as to move as one while maintaining a constant orientation relative to each other.

As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, left, right, upper, lower, front, back, on top of, and derivatives thereof, relate to the orientation of the elements shown in the drawings and are not limiting upon the claims unless expressly recited therein.

Many modern fabrics are woven out of thermoplastic fibers (such as nylon and polyester). By selectively melting fibers within a piece of such fabric, or more particularly selected portions of fibers therein, material properties (e.g., stiffness) of a portion, portions, or the entirety of a single piece of fabric can be selectively manipulated or tailored for a particular application. When such fibers, or portions thereof, are melted the melted portions fuse with adjacent fibers generally melding them together. Such melded areas create low stretch regions within a piece of fabric that may be utilized in various ways, some examples of which are discussed below. When created with a precise technology such as via a laser (melting the fibers using heat) or a chemical screen-printing process (melting the fibers chemically), such low stretch or essentially no stretch regions can be very small (e.g., without limitation, 1-2 mm) and well controlled. These low stretch regions may be combined at the macro scale to create “stretch control patterns” which are patterns that control the stretch, or lack thereof (i.e., stiffness) of a single piece of fabric, or a selected region or regions within a single piece of fabric. Due to the methods by which such regions may be formed, several of such regions, each imparting different deformation properties to the fabric, may be formed within a single piece of fabric. Additionally, such approach can be applied to multi-layer laminate materials wherein selective portions of an outside layer of the laminate can be selectively melted and thus melded onto other fibers within that outside layer or to portions of other layers within the laminate.

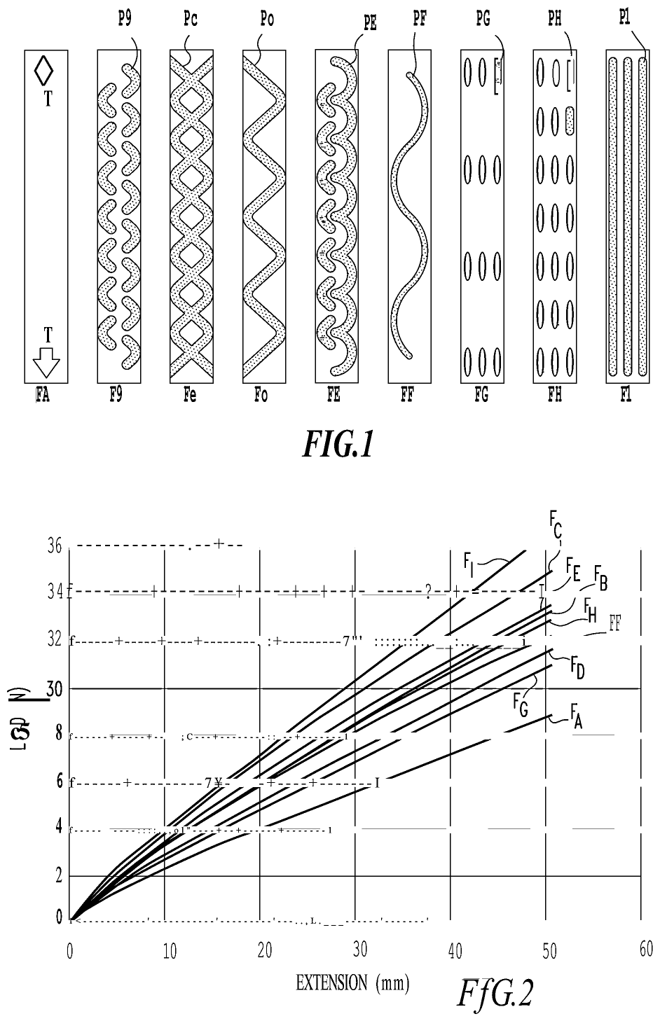

Schematic representations of several different example fabric specimens F_{A}-F_{I} in accordance with example embodiments of the present invention are illustrated in FIG. 1. Selected portions of fabric specimens F_{B}-F_{I}, such as shown by dotted areas, were selectively stiffened via the previously described melting to form one-dimensional stretch control patterns P_{B}-P_{I}. Specimen F_{A} was not selectively stiffened (and thus does not include any dotted regions). FIG. 2 shows a graph of the extension vs. load of each of specimens F_{A}-F_{I} resulting from a tensile force (such as shown by the arrows T on specimen A) applied to each specimen A-I. As can be seen from the graphs of FIG. 2, each of the different stretch control patterns P_{B}-P_{I} of specimens F_{B}-F_{I} formed by selectively stiffened portions thereof provide for a different stiffness (i.e., Load/Extension) in the direction in which tensile force T is applied.

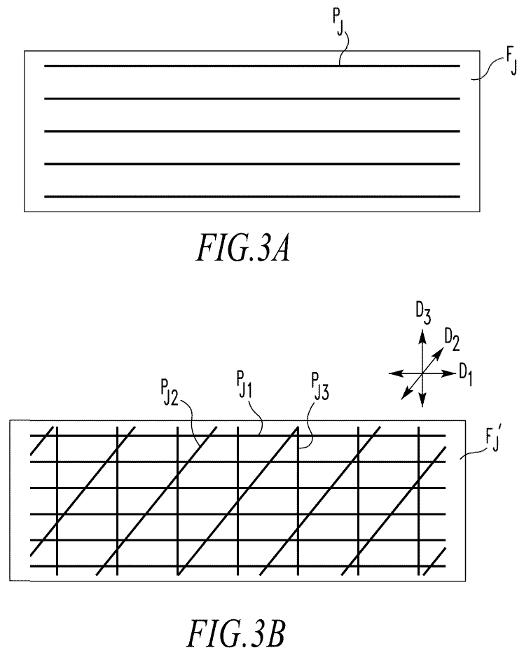

In addition to varying/tailoring stiffness of the fabric in a single direction, multiple one dimensional stretch control patterns oriented at different angles may be combined such that stretch properties of a piece of fabric in several different directions may be controlled. For example, FIG. 3A illustrates another fabric specimen Fⱼ having another one-dimensional stretch control pattern P_{J} in accordance with one example embodiment of the present invention that is similar to stretch pattern P_{I} of specimen F_{I} of FIG. 1. FIG. 3B shows a fabric specimen F_{J}′ in which stretch pattern P_{J} has generally been formed three times (hence labeled P_{J1}, P_{J2}, P_{J3}), each oriented in a different direction, such as shown by double sided arrows D₁, D₂ and D₃. As a result of such arrangement the stiffness of fabric specimen F_{J}′ has been increased in directions D₁, D₂, and D₃.

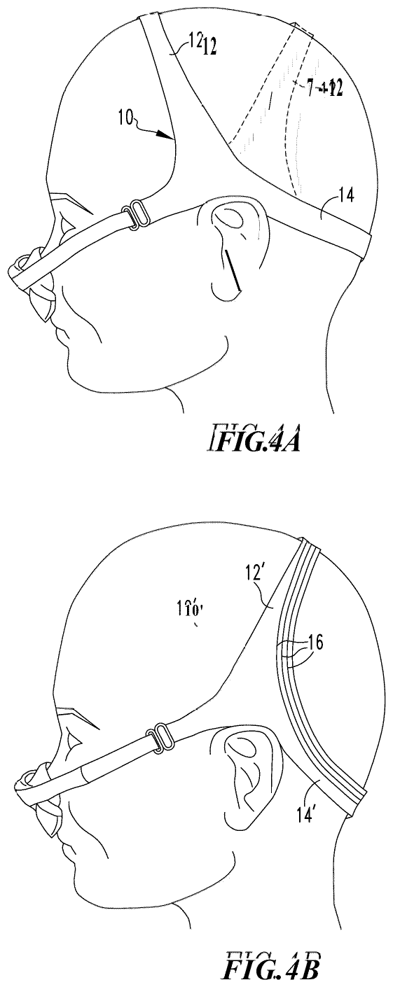

Such techniques can be readily employed in making headgear for use in securing a mask to the head of a patient that improves upon conventional approaches as it is much more cost effective to use a single piece of fabric, which has multiple stretch properties created in accordance with the concepts disclosed herein, than to use multiple pieces of fabric, such as previously discussed in the Background section hereof. As an example, FIG. 4A shows a fabric headgear arrangement 10 positioned on the head of a patient. Headgear arrangement 10 includes a top strap portion 12 and a rear strap portion 14 formed from a unitary piece of fabric material that does not include any stiffened portions such as described herein. Absent any stiffened portions, headgear arrangement 10 tends to readily distort from a preferred positioning (such as shown partially in dashed line), wherein top strap 12 is disposed generally in the upper portion of the rear of the patient's head, to a generally unstable positioning near the top/front of the patient's head. By creating (i.e., melting) stretch control lines 16 that extend along both top and rear strap portions 12′ and 14′ such as shown in FIG. 4B, headgear arrangement 10′ resists distorting such as previously described in conjunction with FIG. 4A, and thus remains properly positioned on the patient's head.

Stiffened areas such as described herein may also be employed to control the way a portion or portions of fabric deflect(s) under tension. There is a myriad of applications in CPAP mask headgear in which it is desirable to apply tension along a vector that does not contain material. For example, many headgears include stiffeners that route the headgear around the ears or eyes of a patient. Embodiments of the present concept can be used to mimic such arrangements using selective melt patterns without a plastic stiffener. By melting a curve whose concavity is opposite that of the desired post-tension shape of the fabric strap, a straight strap that becomes curved when put under tension can be created. Such an arrangement creates an effect similar to that of a stiffened curve such as conventionally employed, while eliminating the need for a plastic core.

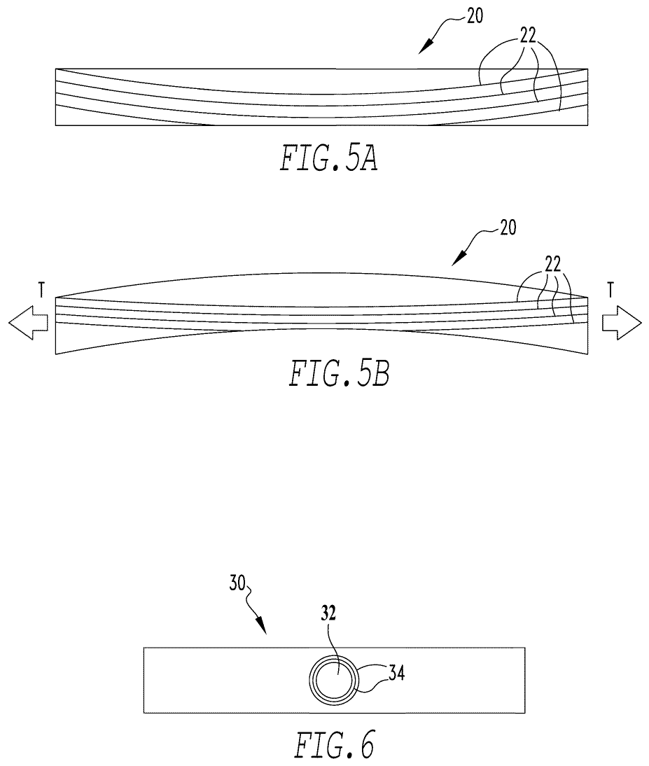

An example arrangement in accordance with one example embodiment of the present invention demonstrating such concept is shown in FIGS. 5A and 5B. More particularly, FIG. 5A shows an example fabric strap 20 in a relaxed (i.e., no force applied) position. Strap 20 includes a plurality of arcuate shaped melt lines 22 with an upward facing concavity. As shown in FIG. 5B, when a tensile force T is applied to strap 20, melt lines 22 tend to straighten as a result of being stiffer than the surrounding material. Such straightening of melt lines 22 causes strap so to generally be pulled in the direction of the concavity of melt lines 22, the resulting in the curved shape shown in FIG. 5B. The characteristics of the lateral deflection of strap 20 can be controlled by the design of the melt pattern. In general, melt regions which have a greater length L to width W ratio tend to result in more deflection, and wider regions create a larger deflection-inducing “force”.

Stiffened areas such as described herein may also be employed adjacent apertures in fabrics. For example, FIG. 6 shows a portion of a fabric strap 30 having a circular aperture 32 defined therethrough. Aperture 32 is encircled by two melt lines 34 which create a stiff ring around aperture 32 to help maintain the shape of aperture 32. This can be done to strengthen a hole to accept some feature like a plastic collar.

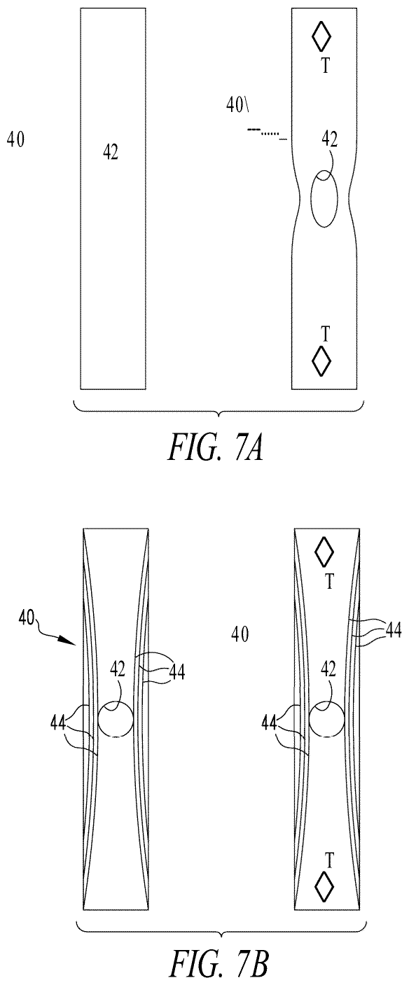

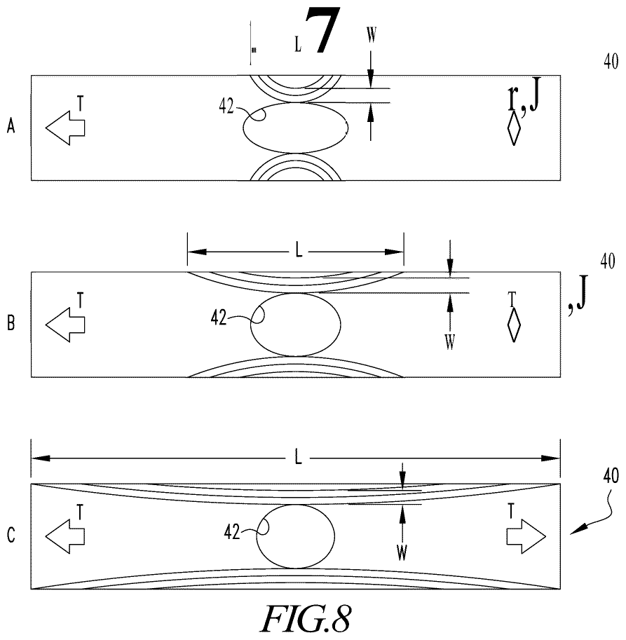

As another example, stiffened areas can be used to prevent buckling at or about an aperture resulting from the Poisson-effect. As shown in FIG. 7A, due to the Poisson Effect (materials in tension tend to contract in directions transverse to said tension) an aperture 42 in a piece of fabric 40 under tensile force T tends to collapse. The Poisson “Force” squeezes aperture 42 from the sides and since aperture 42 lacks material to resist such compression it collapses. As shown in FIG. 7B, by utilizing arcuate shaped stiffened portions 44, similar to those previously discussed in regard to FIGS. 5A and 5B formed on either side of aperture 42, the Poisson-effect can be mitigated. By changing the length of these deflection control features, it is possible to control the buckled profile of aperture 42. Portions A, B and C of FIG. 8 generally illustrate how as the length L of the deflection control features get longer, the final shape of aperture 42 becomes more circular.

From the foregoing examples, it is thus to be appreciated that embodiments of the present invention provide methods of modifying one or more deformation characteristics of a polymer-based fabric material. Such modified fabric materials may then be readily used for making items, such as headgear for use in securing a patient interface device to the head of a patient. Some benefits of selective stiffening such as described herein over current technology are: more options for types of stretch control (i.e. one way stretch control, two way stretch control, hole support, deflection control); finer resolution of stress control features (can create stretch control features on the millimeter scale instead of needing to sew on another large piece of fabric for each one); and the ability to make a variable-stretch headgear out of a single piece of fabric (may lead to cost savings).

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

What is claimed is:

1. A patient interface assembly comprising:
(a) a mask component comprising:
(1) a first portion that contacts a portion of a face of a user responsive to the patient interface assembly being donned by such user, and
(2) a second portion that is adapted to be coupled a supply of gas such that a flow of gas is delivered to an airway of a patient responsive to the patient interface assembly being donned by such user and gas being delivered to the mask component; and
(b) a headgear assembly, comprising:
a first headgear strap having (i) a first end coupled to a first side of the mask component, (ii) a second end couped to a second side of the mask component generally opposite the first side of the mask component, and (iii) a lateral portion extending between the first end and the second end, wherein the lateral portion extends around at least a portion of a user's head responsive to the patient interface assembly being donned by such user, and wherein the lateral portion comprises:
a strip of fabric material, wherein a stiffened portion the fabric material is physically altered to have an extension versus load characteristic that differs from a remaining portion of the fabric material, and wherein the stiffened portion of the fabric material is disposed in a lengthwise direction along the lateral portion.

2. The patient interface assembly of claim 1, wherein the fabric material comprises at least one of nylon or polyester fibers.

3. The patient interface assembly of claim 1, wherein physically altering the fabric material includes selectively melting a number of portions of the fabric material in a predetermined pattern.

4. The patient interface assembly of claim 1, wherein the stiffened portion of the fabric material comprises a number of linear portions.

5. The patient interface assembly of claim 1, wherein the stiffened portion of the fabric material comprises a number of arcuate portions.

6. The patient interface assembly of claim 1, wherein the fabric material is a one of a plurality of layers of a laminate material.

7. The patient interface assembly of claim 1, wherein the lateral portion of the first headgear strap includes a second layer of another material laminated to the fabric material.

8. The patient interface assembly of claim 1, further comprising a second headgear strap, wherein the first headgear strap and the second headgear strap are coupled together such that the first headgear strap extends generally over a top of a user's head and the second headgear strap extends generally behind the user's head responsive to the patient interface assembly being donned by such user.

9. The patient interface assembly of claim 8, wherein the second headgear strap includes a first end coupled to a first portion of the first headgear strap, (ii) a second end couped to a second portion of the first headgear strap, and (iii) a second lateral portion extending between the first end of the second headgear strap and the second end of the second headgear strap, wherein the second lateral portion extends around at least a portion of a user's head responsive to the patient interface assembly being donned by such user, and wherein the lateral portion comprises:
a second strip of second fabric material, wherein a stiffened portion the second fabric material is physically altered to have an extension versus load characteristic that differs from a remaining portion of the second fabric material, and wherein the stiffened portion of the second fabric material is disposed in a lengthwise direction along the lateral portion.

10. The patient interface assembly of claim 9, wherein the stiffened portion the fabric material and the stiffened portion the second fabric material are integral with one another.
